(19) United States
(12) Patent Application Publication
(10) Pub. No.: US 2024/0023648 A1
(43) Pub. Date: Jan. 25, 2024

(54) Masks, Mask Systems, and Methods for Inhibiting Transmission of Droplets and Aerosols During a Medical Procedure

(71) Applicant: Entellus Medical, Inc., Maple Grove, MN (US)

(72) Inventors: Paul Lesch, Lino Lakes, MN (US); Aaron Pidde, Bloomington, MN (US); John Drontle, Buffalo, MN (US)

(21) Appl. No.: 18/044,912

(22) PCT Filed: Sep. 7, 2021
(86) PCT No.: PCT/US2021/049271

§ 371 Date: Mar. 10, 2023

Related U.S. Application Data
(60) Provisional application No. 63/078,309, filed on Sep. 14, 2020.

(51) Int. Cl. A41D 13/11

(52) U.S. Cl. CPC A41D 13/11

(57) ABSTRACT

In an example, a mask includes a containment wall, a gasket, a nasal access in the containment wall, an inlet, and an outlet. The containment wall includes an inner surface, an outer surface opposite the inner surface, and a peripheral edge at an interface between the inner surface and the outer surface. The gasket is at the peripheral edge of the containment wall. The gasket can conform to the face of a wearer. The containment wall and the gasket define a chamber between the inner surface of the containment wall and the face of the wearer when the mask is worn by the wearer. The nasal access opening is aligned with a nose of the wearer when the mask is worn by the wearer. The inlet can receive a gas into the chamber. The outlet can exhaust out of the chamber the gas and aerosols exhaled by the wearer.

### CROSS-REFERENCE TO RELATED APPLICATIONS

The present disclosure claims the benefit of U.S. Provisional Application No. 63/078,309, filed Sep. 14, 2020, the contents of which is hereby incorporated by reference in its entirety.

### FIELD

The present disclosure generally relates to patient-worn surgical masks and, in particular, to masks, systems, and methods for inhibiting transmission of droplets and/or aerosols during a medical procedure.

### BACKGROUND

During medical procedures, medical practitioners universally use face masks to inhibit or prevent the spread of viruses and infectious bacteria between the medical practitioners and a patient. For example, many viruses (e.g., Sars-CoV-2) are spread through droplets and aerosols that are exhaled by an infected person and subsequently inhaled by others within a vicinity of the infected person. When worn by an uninfected person, a mask can provide a physical barrier that can help to inhibit or prevent that person from inhaling the droplets and aerosols. When worn by an infected person, a mask can provide a physical barrier that can inhibit or block the outward transmission of the droplets and aerosols, and thereby reduce a risk that an uninfected person will inhale the droplets and aerosols.

### SUMMARY

In an example, a mask for performing a medical procedure via a nasal cavity is described. The mask includes a containment wall, a gasket, a nasal access in the containment wall, an inlet at a superior end of the containment wall, and an outlet at an inferior end of the containment wall. The containment wall includes an inner surface configured to extend over a face of a wearer, an outer surface opposite the inner surface, and a peripheral edge at an interface between the inner surface and the outer surface. The gasket is at the peripheral edge of the containment wall. The gasket is configured to conform to the face of a wearer. The containment wall and the gasket define a chamber between the inner surface of the containment wall and the face of the wearer when the mask is worn by the wearer. The nasal access opening is aligned with a nose of the wearer when the mask is worn by the wearer. The inlet is configured to receive a gas into the chamber. The outlet is configured to exhaust out of the chamber the gas and aerosols exhaled by the wearer while wearing the mask.

In another example, a surgical mask system includes a mask and a pump. The mask includes a containment wall including: (i) an inner surface configured to extend over a face of a wearer, (ii) an outer surface opposite the inner surface, and (iii) a peripheral edge at an interface between the inner surface and the outer surface. The mask also includes a gasket at the peripheral edge of the containment wall. The gasket is configured to conform to the face of a wearer. The containment wall and the gasket define a chamber between the inner surface of the containment wall and the face of the wearer when the mask is worn by the wearer. The mask further includes a nasal access opening in the containment wall. The nasal access opening is aligned with a nose of the wearer when the mask is worn by the wearer. Additionally, the mask includes an inlet at a superior end of the containment wall and an outlet at an inferior end of the containment wall. The inlet is configured to receive a gas into the chamber, and the outlet is configured to exhaust out of the chamber the gas and aerosols exhaled by the wearer while wearing the mask. The pump is coupled to the outlet of the mask. The pump is configured to provide suction at the outlet.

In another example, a surgical mask system is described. The surgical mask system includes a mask, an exhaust tube, and a pump. The mask includes a mask wall configured to be positioned over a lower portion of a face of a wearer below a nose of the wearer. The mask wall defines a cavity between the mask wall and the face of the wearer when the mask is worn by the wearer. The mask also includes an intake at a superior end of the mask wall, and an outlet at an inferior end of the mask wall. The intake is configured to receive air and aerosols exhaled by the wearer into the cavity, and the outlet is configured to exhaust the air and the aerosols out of the cavity. The exhaust tube is coupled to the outlet of the mask. The pump is coupled to the exhaust tube of the mask. The pump is configured to provide suction at the outlet to draw the air and the aerosols into the cavity of the mask.

In another example, a method of forming a mask for performing a medical procedure via a nasal cavity is described. The method can include forming a containment wall. The containment wall can include: (i) an inner surface configured to extend over a face of a wearer, (ii) an outer surface opposite the inner surface, (iii) a peripheral edge at an interface between the inner surface and the outer surface, and (iv) a nasal access opening that is configured to be aligned with a nose of the wearer when the mask is worn by the wearer.

The method can also include forming a gasket at the peripheral edge of the containment wall. The gasket can be configured to conform to the face of a wearer. The containment wall and the gasket define a chamber between the inner surface of the containment wall and the face of the wearer when the mask is worn by the wearer. Additionally, the method can include forming an inlet at a superior end of the containment wall, wherein the inlet is configured to receive a gas into the chamber. The method can also include forming an outlet at an inferior end of the containment wall. The outlet is configured to exhaust out of the chamber (i) the gas and (ii) at least one of droplets or aerosols exhaled by the wearer while wearing the mask.

In another example, a method of operating a surgical mask system is described. The method can include positioning a mask on a face of a patient. The mask can include a containment wall, a gasket, a nasal access in the containment wall, an inlet at a superior end of the containment wall, and an outlet at an inferior end of the containment wall. The containment wall can include an inner surface that extends over a face of a wearer, an outer surface opposite the inner surface, and a peripheral edge at an interface between the inner surface and the outer surface. The gasket is at the peripheral edge of the containment wall. The containment wall and the gasket define a chamber between the inner surface of the containment wall and the face of the wearer. The nasal access opening is aligned with a nose of the wearer. The inlet is configured to receive a gas into the chamber. The outlet is configured to exhaust out of the chamber the gas along with droplets and/or aerosols exhaled by the wearer while wearing the mask.

The method can also include using a pump to provide suction at the outlet and cause a laminar flow of the gas between the inlet and the outlet. Additionally, the method includes directing, using the laminar flow of the gas, the droplets and/or the aerosols away from the nasal access opening and towards the outlet. The method further includes exhausting through the outlet the gas along with the droplets and/or the aerosols from the chamber.

The features, functions, and advantages that have been discussed can be achieved independently in various embodiments or may be combined in yet other embodiments further details of which can be seen with reference to the following description and drawings.

### BRIEF DESCRIPTION OF THE FIGURES

The novel features believed characteristic of the illustrative embodiments are set forth in the appended claims. The illustrative embodiments, however, as well as a preferred mode of use, further objectives and descriptions thereof, will best be understood by reference to the following detailed description of an illustrative embodiment of the present disclosure when read in conjunction with the accompanying drawings, wherein:

FIG. 1 illustrates a simplified block diagram of a surgical mask system, according to an example.

FIG. 2A illustrates a perspective view of a mask for use with the surgical mask system shown in FIG. 1, according to an example.

FIG. 2B illustrates another perspective view of the mask shown in FIG. 2A, according to an example.

FIG. 2C illustrates a side view of the mask shown in FIG. 2A positioned on a face of a wearer, according to an example.

FIG. 3 illustrates an implementation of the surgical mask system shown in FIG. 1, according to another example.

FIG. 4 illustrates an implementation of the surgical mask system shown in FIG. 1, according to another example.

FIG. 5 illustrates an implementation of the surgical mask system shown in FIG. 1, according to another example.

FIG. 6A illustrates an implementation of the mask shown in FIG. 1 in a first position, according to another example.

FIG. 6B illustrates an implementation of the mask shown in FIG. 1 in a second position, according to another example.

FIG. 7 illustrates a surgical mask system, according to another example.

FIG. 8 illustrates a flowchart for a process of operating a surgical mask system is depicted according to an example.

FIG. 9 illustrates a flowchart for a process of operating a surgical mask that can be used with the process shown in FIG. 8, according to an example.

FIG. 10 illustrates a flowchart for a process of operating a surgical mask that can be used with the process shown in FIG. 8, according to an example.

FIG. 11 illustrates a flowchart for a process of operating a surgical mask that can be used with the process shown in FIG. 8, according to an example.

FIG. 12 illustrates a flowchart for a process of operating a surgical mask that can be used with the process shown in FIG. 11, according to an example.

FIG. 13 illustrates a flowchart for a process of operating a surgical mask that can be used with the process shown in FIG. 8, according to an example.

FIG. 14 illustrates a flowchart for a process of operating a surgical mask that can be used with the process shown in FIG. 13, according to an example.

FIG. 15 illustrates a flowchart for a process of operating a surgical mask that can be used with the process shown in FIG. 13, according to an example.

FIG. 16 illustrates a flowchart for a process of operating a surgical mask that can be used with the process shown in FIG. 8, according to an example.

FIG. 17 illustrates a flowchart for a process of operating a surgical mask that can be used with the process shown in FIG. 16, according to an example.

FIG. 18A illustrates a perspective view of an outer surface of a mask including a membrane having a plurality of layers, according to an example.

FIG. 18B illustrates a perspective view of an inner surface of the mask shown in FIG. 18A, according to an example.

FIG. 19A illustrates a top view of the outer surface of the membrane shown in FIG. 18A, according to an example.

FIG. 19B illustrates a cross-sectional of the membrane taken through a line shown in FIG. 19A, according to an example.

FIG. 19C illustrates a partial perspective view of the layers of the membrane shown in FIG. 19 with a membrane gasket removed, according to an example.

FIG. 20 illustrates a flowchart for a process of forming a surgical mask, according to an example.

FIG. 21 illustrates a flowchart for a process of forming a surgical mask that can be used with the process shown in FIG. 20, according to an example.

FIG. 22 illustrates a flowchart for a process of forming a surgical mask that can be used with the process shown in FIG. 20, according to an example.

FIG. 23 illustrates a flowchart for a process of forming a surgical mask that can be used with the process shown in FIG. 22, according to an example.

FIG. 24 illustrates a flowchart for a process of forming a surgical mask that can be used with the process shown in FIG. 23, according to an example.

FIG. 25 illustrates a flowchart for a process of forming a surgical mask that can be used with the process shown in FIG. 23, according to an example.

FIG. 26 illustrates a flowchart for a process of forming a surgical mask that can be used with the process shown in FIG. 22, according to an example.

FIG. 27 illustrates a flowchart for a process of forming a surgical mask that can be used with the process shown in FIG. 26, according to an example.

FIG. 28 illustrates a flowchart for a process of forming a surgical mask that can be used with the process shown in FIG. 22, according to an example.

FIG. 29 illustrates a flowchart for a process of forming a surgical mask that can be used with the process shown in FIG. 22, according to an example.

FIG. 30 illustrates a flowchart for a process of forming a surgical mask that can be used with the process shown in FIG. 20, according to an example.

### DETAILED DESCRIPTION

Disclosed embodiments will now be described more fully hereinafter with reference to the accompanying drawings, in which some, but not all of the disclosed embodiments are shown. Indeed, several different embodiments may be described and should not be construed as limited to the embodiments set forth herein. Rather, these embodiments are described so that this disclosure will be thorough and complete and will fully convey the scope of the disclosure to those skilled in the art.

By the term “approximately” or “substantially” with reference to amounts or measurement values described herein, it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

As noted above, during medical procedures, medical practitioners generally use face masks to inhibit or prevent the spread of viruses and infectious bacteria between the medical practitioners and a patient. Typically, medical practitioners wear the face masks while the patient does not. This is particularly the case in the context of medical procedures that require access to the patient's nasal cavity as conventional face masks fully cover the patient's nose and mouth to provide a physical barrier for limiting the transmission of droplets and/or aerosols (e.g., liquid aerosols and/or solid aerosols). However, while a risk of viral and/or bacterial transmission can be reduced when only the medical practitioners wear masks, this risk can be reduced further when the patient also wears a mask.

The present disclosure provides a mask, a mask system, and methods that can inhibit the transmission of droplets and aerosols exhaled by a wearer during a medical procedure. For instance, within examples of the present disclosure, a mask, a mask system, and a method can provide a laminar flow of a gas over a nose and a mouth of a patient while providing access to a nasal cavity of the patient through a nasal access opening in a containment wall of the mask. The laminar flow of the gas can cause the droplets and the aerosols exhaled by the wearer to be directed away from the nasal access opening and toward an outlet in the mask, which can safely divert the droplets and/or the aerosols away from the medical practitioners performing the medical procedure on the wearer. As a result, the masks, the mask systems, and methods of the present disclosure can allow the medical practitioners to perform the medical procedure using one or more surgical tools extending through the nasal access opening while mitigating a risk of transmitting the droplets and/or the aerosols through the nasal access opening from the patient to the medical practitioners.

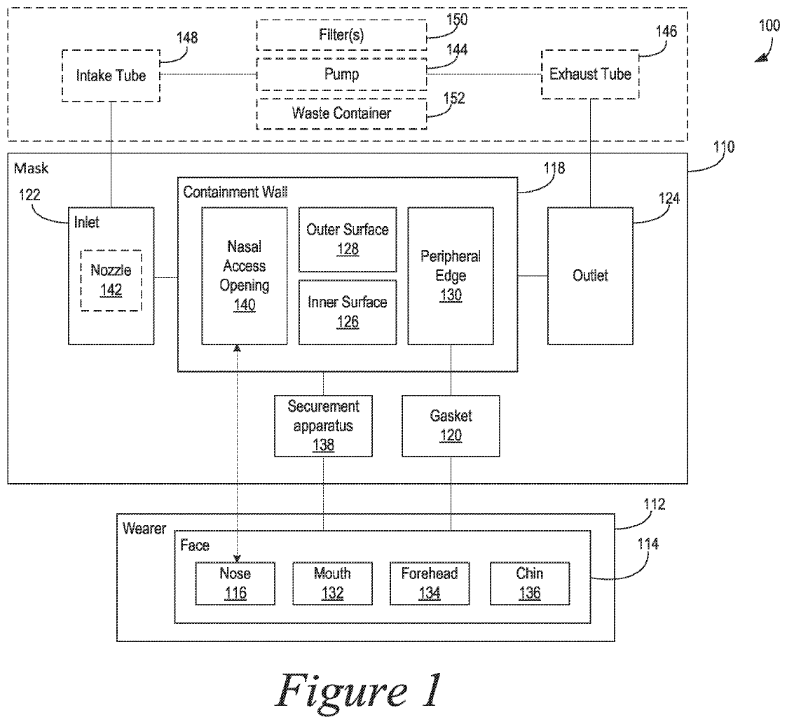

Referring to FIG. 1, a simplified block diagram of a surgical mask system 100 is shown according to an example. As shown in FIG. 1, the surgical mask system 100 includes a mask 110 that can be coupled to a face 114 of a wearer 112 to inhibit transmission of droplets and/or aerosols that may be exhaled by the wearer 112 during a medical procedure.

The wearer 112 can be a patient and/or a client of one or more medical practitioners, and the medical procedure can involve one or more medical practitioners inserting one or more instruments into a nasal cavity of a nose 116 of the wearer 112. As examples, the medical procedure can include one or more procedures selected from among a group of procedures consisting of: ablating a tissue and/or a nerve in the nasal cavity (e.g., using a cryotherapy modality, a radiofrequency electric current modality, a microwave modality, an ultrasonic modality, and/or a laser light modality), implanting a nasal implant (e.g., to treat a nasal valve collapse, support nasal cartilage, and/or improve breathing), performing a sinuplasty procedure (e.g., a balloon catheter dilation procedure), performing an Eustachian tube dilation tube procedure (e.g., a balloon catheter dilation procedure), performing an ethmoidectomy, performing a polypectomy, performing a septoplasty, removing a tumor, to reduce a size of a nasal turbinate, and/or performing a rhinoplasty.

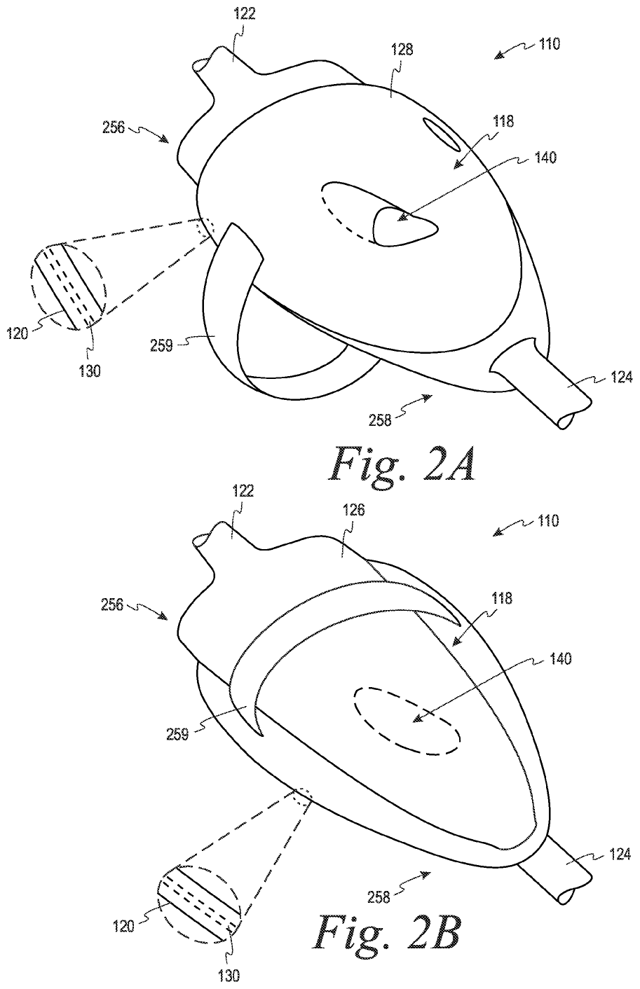

As shown in FIG. 1, the mask 110 includes a containment wall 118, a gasket 120, an inlet 122, and an outlet 124. The containment wall 118 includes an inner surface 126 configured to extend over the face 114 of the wearer 112, an outer surface 128 opposite the inner surface 126, and a peripheral edge 130 at an interface between the inner surface 126 and the outer surface 128. The gasket 120 is at the peripheral edge 130 of the containment wall 118. In this arrangement, the gasket 120 can contact the face 114 while at least a portion of the inner surface 126 can be separated from the face 114 by a gap when the mask 110 is positioned on the face 114 of the wearer 112. As a result, the containment wall 118 and the gasket 120 define a chamber (shown in FIGS. 2A-2B) between the inner surface 126 of the containment wall 118 and the face 114 of the wearer 112 when the mask 110 is worn by the wearer 112.

As shown and described in further detail below with respect to FIGS. 2C-5, when the mask 110 is positioned over the face 114 of the wearer 112, at least the nose 116 and a mouth 132 of the wearer 112 is positioned within the chamber between the containment wall 118 and the face 114 of the wearer 112. In this way, the containment wall 118 can provide a physical barrier that inhibits or substantially prevents the droplets and/or the aerosols from passing through the containment wall 118 from chamber to an external environment in which the medical practitioners may be located. This in turn can help to reduce a risk of transmission of an infectious virus and/or bacteria contained in the aerosols from the wearer 112 to the medical practitioners.

To inhibit or substantially prevent the aerosols passing through the containment wall 118, the containment wall 118 can be entirely non-permeable with respect to the droplets and/or the aerosols exhaled by the wearer 112. For instance, the containment wall 118 can be formed from a non-porous material and/or a material having a pore size that is smaller than a size of the droplets and/or a size of the aerosols. In one implementation, the containment wall 118 can be made from one or more materials such that the containment wall 118 has a pore size that is less than or equal to approximately 0.125 microns, which is the approximate size of aerosols associated with the Sars-CoV-2 virus. In another implementation, the containment wall 118 can be made from one or more materials such that the containment wall 118 has a pore size between approximately 0.125 microns and approximately 0.30 microns. In this implementation, the containment wall 118 can also inhibit or prevent transmitting the Sars-CoV-2 virus because the Sars-CoV-2 virus typically attaches to particulate matter when airborne such that the Sars-CoV-2 virus and the particulate matter have a combined size that is greater than approximately 0.30 microns. In other examples, the pore size of the containment wall 118 can be greater than 0.30 microns where other, larger types of droplets and/or aerosols are to be inhibited and/or prevented from transmission by the mask 110.

In other examples, the containment wall 118 can be substantially non-permeable with respect to the droplets and/or the aerosols exhaled by the wearer 112. As used herein, the term “substantially non-permeable” means the containment wall 118 prevents the transmission of at least 95 percent of the droplets and/or the aerosols.

In some examples, the containment wall 118 can be formed from one or more materials that cause the containment wall 118 to be at least partially translucent (e.g., transparent). This can, for instance, allow the medical practitioner to see a position of the surgical instruments relative to the nose 116 of the wearer 112 as the medical practitioner performs the medical procedure. Forming the containment wall 118 from the one or more materials that cause the containment wall 118 to be at least partially translucent or transparent can additionally or alternatively allow the medical practitioner to visually perceive transdermal illumination provided by a surgical instrument to navigate and/or confirm a position of the surgical instrument within the nasal cavity. Additionally or alternatively, forming the containment wall 118 from the one or more materials that cause the containment wall 118 to be at least partially translucent or transparent can help enhance wearer comfort (e.g., in implementations in which the wearer may be awake during the medical procedure) by allowing the wearer to see through the containment wall 118.

In some examples, the containment wall 118 can be formed from a relatively rigid material. This can help to maintain the containment wall 118 in a fixed shape. As described in further detail below, the shape of the containment wall 118 can be a factor that affects a flow of a gas within the chamber and helps to control the droplets and/or the aerosols within the chamber. Example materials that can provide the containment wall 118 with at least one of the permeability, transparency, and/or rigidity described above include one or more materials selected from among a group of materials including: a polymer material, a thermoplastic material (e.g., a polycarbonate material, acrylic, styrene, and/or polyethylene terephthalate), a thermoset material, and/or glass.

As noted above, the gasket 120 can contact the face 114. To help mitigate transmitting the droplets and/or the aerosols egressing out of the chamber at an interface between the gasket 120 and the face 114, the gasket 120 can be configured to conform to the face 114 of the wearer 112. For example, the gasket 120 can be formed from a deformable material, have a size, and/or have a shape that allows the gasket 120 to compress against and/or conform to the face 114 of the wearer 112. For instance, in an implementation, the gasket 120 can include a material that is less rigid than a material of the containment wall 118 such as, for example, a soft elastomeric material (e.g., a silicone elastomer). The compressibility and/or rigidity of the gasket 120 can additionally or alternatively allow the mask 110 to be compatible with a greater range of face shapes and/or face sizes of wearers. The gasket 120 can additionally or alternatively provide cushioning that enhances the comfort of the wearer 112 while wearing the mask 110.

In some examples, the gasket 120 can be configured to provide a seal between the containment wall 118 and the face 114 of the wearer 112. For instance, the gasket 120 can be in relatively continuous contact with the face 114 of the wearer 112 over most or an entirety of the peripheral edge 130 of the containment wall 118. This can help to form a pressure-tight seam between the peripheral edge 130 and the face 114, which inhibits or prevents transmission of the droplets and/or the aerosols into and/or out of the chamber between the mask 110 and the face 114 of the wearer 112.

In some examples, the gasket 120 and the containment wall 118 can be separate structures that are coupled to each other. For instance, the gasket 120 can be coupled to the peripheral edge 130 of the containment wall 118 by welding, adhesive attachment, threaded engagement, interlocking engagement, and/or frictional engagement. In other examples, the gasket 120 and the containment wall 118 can be integrally formed as a unitary, monolithic structure. For instance, the gasket 120 can be formed of with relatively smaller thickness (e.g., in a dimension extending between the inner surface 126 and the outer surface 128) than a thickness of the containment wall 118 such that the gasket 120 can compress against and/or conform to the face 114 as described above.

In some examples, the gasket 120 can extend around an entirety of the peripheral edge 130 of the containment wall 118. This can help to provide the seal around the entirety of the peripheral edge 130 of the containment wall 118. However, in other examples, the gasket 120 can extend along only a portion of the peripheral edge 130. For instance, the gasket 120 may be omitted along a portion of the peripheral edge 130 that is upstream of a flow of gas through the chamber (as described in further detail below).

As noted above, when the mask 110 is positioned over the face 114 of the wearer 112, the mask 110 extends over the mouth 132 and at least a portion of the nose 116 of the wearer 112. In some examples, the mask 110 can additionally extend over at least a portion of a forehead 134 and/or at least a portion of a chin 136 of the wearer 112. As described in further detail below, this can help to provide a flow path for a gas flowing over the nose 116 and the mouth 132 to control and capture the droplets and/or the aerosols exhaled by the wearer 112 during the medical procedure.

As shown in FIG. 1, the mask 110 can additionally include a securement apparatus 138 that can couple the mask 110 to the face 114 of the wearer 112. As one example, the securement apparatus 138 can include a strap that extends from a first lateral side of the containment wall 118 to a second lateral side of the containment wall 118. In this arrangement, the strap can extend around a back of a head of the wearer 112 such that the strap can couple the mask 110 to the head of the wearer 112 (e.g., by maintaining the mask 110 in a desired position on the face 114 of the wearer 112).

In an example, the strap can be formed from an elastic material. This can allow the strap to assist in applying pressure to the mask 110 to compress and conform the gasket 120 to the face 114 of the wearer 112 and, thus, help to provide a seal between the gasket 120 and the face 114 of the wearer 112. In another example, the strap can additionally or alternatively include a ratchet mechanism that provides for adjusting a size of the strap to thereby adjust an amount of pressure between the gasket 120 and the face 114 of the wearer 112.

In some examples, the securement apparatus 138 can include a single strap. In other examples, the securement apparatus 138 can include a plurality of straps. Although the strap is described above as being configured to extend around the head of the wearer 112, the securement apparatus 138 can additionally or alternatively include one or more straps that do not extend around the head of the wearer 112. For instance, the securement apparatus can include one or more straps that couple to a patient procedure chair, a table, and/or a bed via one or more fasteners (e.g., metal tacks, rivets, buttons, magnets, hook and loop fasteners, buckles, and/or snaps).

The securement apparatus 138 can additionally or alternatively include an adhesive that couples the gasket 120 to the face 114 of the wearer 112. For example, the adhesive can be a bio-compatible adhesive such as, for instance, a medical tape, a foam tape, and/or other skin-friendly adhesives. In one example, the bio-compatible adhesive can be a foam material. This can help to provide and/or enhance the sealing properties of the gasket 120.

As shown in FIG. 1, the mask 110 also includes a nasal access opening 140 in the containment wall 118. The nasal access opening 140 extends entirely through the containment wall 118 between the inner surface 126 and the outer surface 128 such that the medical practitioner can insert one or more instruments through the nasal access opening 140 to operate on the nose 116 and/or in a nasal cavity of the wearer 112. At the nasal access opening 140, an edge of containment wall 118 may constrain a range of motion of the instrument extending through the nasal access opening 140. To enhance the range of motion, the nasal access opening 140 can be aligned with the nose 116 of the wearer 112 when the mask 110 is worn by the wearer 112, as described in further detail below with respect to FIGS. 2C-4.

In one example, the nasal access opening 140 can have a size of approximately 2 square centimeters to approximately 8 square centimeters. This size can provide a large enough opening to allow the medical practitioner to access and operate the instrument extending through the nasal access opening 140 yet small enough to substantially mitigate leakage of the droplets and/or the aerosols out of the chamber through nasal access opening 140. However, the nasal access opening 140 can have other sizes in other examples.

To prevent or reduce an amount of the droplets and/or aerosols egressing from the chamber at the nasal access opening 140, the mask 110 is configured to provide a laminar flow of a gas (e.g., air) along the inner surface 126 and across the nasal access opening 140. For example, in FIG. 1, the inlet 122 can receive the gas into the chamber and the outlet 124 can exhaust out of the chamber the gas along with droplets and/or the aerosols exhaled by the wearer 112 while wearing the mask 110. Additionally, the inlet 122 can be located on one side of the nasal access opening 140 and the outlet 124 can be located an opposite side of the nasal access opening 140. For instance, the inlet 122 can be at a superior end of the containment wall 118 (e.g., near the forehead 134) and the outlet 124 can be at an inferior end of the containment wall 118 (e.g., near the chin 136). Additionally, for instance, the laminar flow of the gas can be over at least a central longitudinal portion of the containment wall 118 such that the gas flows over the nose 116 and the mouth 132 of the wearer 112 when the wearer 112 wears the mask 110.

In this arrangement, the gas (i) enters the chamber through the inlet 122, (ii) moves with a laminar flow in an inferior direction through the chamber, (iii) flows across the nasal access opening 140 while directing any droplets and/or the aerosols exhaled by the nose 116 in the inferior direction away from the nasal access opening 140, (iv) flows over the mouth 132 while directing any droplets and/or the aerosols exhaled by the nose 116 in the inferior direction away from the nasal access opening 140, and (v) exhausts out of the chamber through the outlet 124. As described in further detail below, the outlet 124 can be coupled to an apparatus that can provide for safe containment and/or neutralize a viral and/or a bacterial content of the droplets and/or the aerosols. In this way, the mask 110 can controllably divert the droplets and/or the aerosols exhaled by the wearer 112 away from the nasal access opening 140 and towards the outlet 124 to reduce or minimize a risk of the patient infecting the medical practitioner that is performing the medical procedure on the wearer 112.

In some examples, the containment wall 118 can assist in providing the laminar flow of the gas between the inlet 122 and the outlet 124. For instance, in an example, the inner surface 126 of the containment wall 118 can have a rounded shape with an apex that is located between the inlet 122 and the nasal access opening 140. The rounded shape of the inner surface 126 can omit any sharp corners and, thus, help to provide a smooth flow path between the inlet 122 and the outlet 124. This laminar flow of the gas can help to more efficiently and effectively guide the droplets and/or the aerosols across the nasal access opening 140 and inhibit the droplets and/or the aerosols from flowing out of the chamber at the nasal access opening 140. The laminar flow of the gas can more efficiently and effectively direct the droplets and/or the aerosols to the outlet 124 than a turbulent flow of the gas.

Additionally or alternatively, the inlet 122 can include a nozzle 142 that can assist in providing the laminar flow of the gas between the inlet 122 and the outlet 124. For example, the nozzle 142 can have cross-sectional dimensions that taper inwardly and reduce in size in a direction from the superior end toward the inferior end. This can help to increase the flow rate of the gas while maintaining a smooth stream of gas at the inlet 122. In some examples, the nozzle 142 can have an elongated shape in a direction between the first lateral side and the second lateral side of the containment wall 118. This can help to spread the flow of gas out over the face 114 of the wearer 112.

Also, in some examples, the nozzle 142 can be positioned and oriented to direct the laminar flow of gas parallel to the inner surface 126 of the containment wall 118. This can help the containment wall 118 to more gradually and smoothly direct the laminar flow of gas along a contour of the inner surface 126 and toward the outlet 124.

As shown in FIG. 1, the mask 110 can be coupled to one or more optional components that can further assist in providing the laminar flow of the gas and/or neutralizing potentially harmful particles (e.g., the droplets and/or the aerosols) from the gas. For instance, as shown in FIG. 1, the mask 110 can be coupled to one or more components selected from a group of components including a pump 144, an exhaust tube 146, an intake tube 148, one or more gas filters 150, and/or one or more waste containers 152.

The pump 144 is coupled to the outlet 124 of the mask 110 by the exhaust tube 146. In this arrangement, the pump 144 is configured to provide suction at the outlet 124. As examples, the pump 144 can be a portable vacuum pump (e.g., on a movable cart) and/or a hospital vacuum system (e.g., an in-wall vacuum system). Additionally, as examples, the pump 144 can be a positive-displacement pump such as, for instance, a rotary-type positive displacement pump (e.g., a gear pump, a screw pump, a rotary vane pump, a hollow disk pump, and/or a vibratory pump), a reciprocating-type positive displacement pump (e.g., a plunger pump, a diaphragm pump, a piston pump, and/or a radial piston pump), and/or a linear-type positive displacement pump. Within examples, the pump 144 can be operable to generate a vacuum pressure between approximately 0 inch of mercury (Hg) and approximately 26 Hg. Additionally, with examples, the pump 144 can be operable to generate a flow rate of the gas that can help to provide the gas with the laminar flow between the inlet 122 and the outlet 124 and, thus, more efficiently and effectively direct the droplets and/or the aerosols to the outlet 124 than a turbulent flow of the gas.

In this arrangement, when the pump 144 provides suction at the outlet 124, the pump 144 can assist in directing to the outlet 124 the laminar flow of gas along with the droplets and/or the aerosols exhaled by the wearer 112. Additionally, by providing suction at the outlet 124, the pump 144 can assist in drawing the gas into the chamber at the inlet 122, and causing the gas to flow over the nose 116 and the mouth 132 of the wearer 112 to the outlet 124.

In some examples, the pump 144 may not be coupled to the inlet 122. However, in other examples, the pump 144 can be additionally or alternatively coupled to the inlet 122 by the intake tube 148. In such examples, the pump 144 can provide a positive flow of gas to the inlet 122 through the intake tube 148. This can help to enhance the laminar flow of the gas between the inlet 122 and the outlet 124. Additionally, using the pump 144 to provide a positive flow of the gas at the inlet 122 can help to reduce the vacuum pressure provided by the pump 144 at the outlet 124 for a given flow rate of the gas within the chamber.

In some examples, the pump 144 can provide to the inlet 122 the positive flow of the gas at a pressure that is less than a pressure of the suction provided by the pump 144 at the outlet 124.

Within examples, the one or more gas filters 150 can remove one or more particles from the gas as the gas is received into the chamber at the inlet 122 and/or as the gas exits the chamber at the outlet 124. As examples, the gas filter 150 can be a high efficiency particulate air (HEPA) filter.

When the gas filter 150 is provided at and/or coupled to the inlet 122, the gas filter 150 can protect the wearer 112 from inhaling potentially harmful particles that may be present in an environment external to the chamber such as, for instance, droplets and/or aerosols that may be exhaled by the medical practitioner. When the gas filter 150 is provided at the outlet 124, the gas filter 150 can help to protect the medical practitioner in the vicinity of the wearer 112 by inhibiting or preventing the droplets and/or the aerosols from passing into the environment in which the medical practitioner is located.

As examples, the gas filter 150 can be located in the pump 144, at an interface between the pump 144 and the exhaust tube 146, along the exhaust tube 146 (e.g., between opposing ends of the exhaust tube 146), at an interface between the exhaust tube 146 and the outlet 124, at an interface between the intake tube 148 and the pump 144, along the intake tube 148 (e.g., between opposing ends of the intake tube 148), and/or at an interface between the intake tube 148 and the inlet 122. Additionally or alternatively, in an implementation that omits the intake tube 148, the inlet 122 can include the gas filter 150.

In some examples, the gas filter 150 can be replaceable. For instance, the gas filter 150 can include an enclosure into which a filter medium (e.g., a filter cartridge) can be inserted, and subsequently removed and replaced with a replacement filter medium (e.g., a replacement filter cartridge). This may be beneficial in implementations in which the mask 110 and/or the pump 144 may be used during a plurality of medical procedures on the same patient or different patients. In other examples, the gas filter 150 can be permanently coupled to the mask 110 and/or the pump 144 such that the gas filter 150 is not replaceable. This may be beneficial, for example, in implementations in which the mask 110 is intended to be a single-use device that is discarded after the medical procedure.

In some examples, the surgical mask system 100 can additionally or alternatively include the one or more waste containers 152. The waste container(s) 152 can be in a vacuum circuit with the pump 144 and the exhaust tube 146 such that the gas flows from the outlet 124 to the waste container(s) 152, which can contain the droplets and/the aerosols in the gas until the waste containers 152 are cleaned, sterilized, and/or discarded. One example of a suitable arrangement for the pump 144 including the one or more waste containers 152 is described in U.S. Pat. No. 7,621,898, the contents of which are hereby incorporated by reference in their entirety. Another example of a suitable arrangement for the pump 144 including the one or more waste containers is the NEPTUNE 3 Waste Management System sold by STRYKER, which is currently headquartered at 2895 Airview Boulevard, Kalamazoo, MI 49002.

FIGS. 2A-6B illustrate implementations of the surgical mask system 100 and/or the mask 110 according to some examples. It should be understood that the example implementations shown in FIGS. 2A-6B can be modified and/or combined with each other to include all or any subset of the features shown and described in FIG. 1.

FIGS. 2A-2C show the mask 110 for performing a medical procedure via a nasal cavity according to an example implementation. In particular, FIG. 2A depicts a perspective view of the outer surface 128 of the mask 110, FIG. 2B shows a perspective view of the inner surface 126 of the mask 110, and FIG. 2C depicts the mask 110 positioned on a face 114 of a wearer 112 (e.g., a patient) and coupled to the pump 144 according to the example implementation.

As shown in FIGS. 2A-2C, the mask 110 includes the containment wall 118 having the inner surface 126 that can extend over the face 114 of the wearer 112, the outer surface 128 opposite the inner surface 126, and the peripheral edge 130 at an interface between the inner surface 126 and the outer surface 128. The mask 110 also includes the gasket 120 at the peripheral edge 130. As described above, the gasket 120 can conform to the face 114 of the wearer 112 and/or provide a seal between the containment wall 118 and the face 114 of the wearer 112.

Additionally, as shown in FIG. 2C, the containment wall 118 and the gasket 120 define a chamber 254 between the inner surface 126 of the containment wall 118 and the face 114 of the wearer 112 when the mask 110 is worn by the wearer 112. As shown in FIGS. 2A-2C, the inlet 122 is at a superior end 256 of the containment wall 118, and the outlet 124 is at an inferior end 258 of the containment wall 118. As shown in FIG. 2C, the superior end 256 of the containment wall 118 extends over the forehead 134 of the wearer 112 and the inferior end 258 of the containment wall 118 extends over the chin 136 of the wearer 112. Additionally, in FIG. 2C, the securement apparatus 138 includes a strap 259 that extends around a head of the wearer 112.

The nasal access opening 140 is in the containment wall 118 at a location between the superior end 256 and the inferior end 258. As described above, the nasal access opening 140 is aligned with the nose 116 of the wearer 112 such that the nasal access opening 140 provides more direct access to the nose and the nasal cavity of the wearer 112 than to the mouth of the wearer 112 when the mask 110 is positioned on the face 114 of the wearer 112. For example, FIG. 2C shows an axis 260 of the nostrils of the wearer 112 (e.g., the external openings to the nasal cavities), and the axis 260 extends through the nasal access opening 140. In this arrangement, the medical practitioner can insert an instrument through the nasal access opening 140 and into a nasal cavity of the wearer 112 with a longitudinal axis of the instrument aligned with and/or approximately parallel to the axis 260 of the nostrils and/or the nasal cavity. This can help to enhance a range of motion for the instrument as the instrument extends through the nasal access opening 140 during the medical procedure.

In FIG. 2C, the pump 144 is coupled to the outlet 124 of the mask 110 by the exhaust tube 146 and the pump 144 is coupled to the inlet 122 by the intake tube 148. As such, the pump 144 can provide suction at the outlet 124 through the exhaust tube 146 and/or the pump 144 can provide a positive flow of gas at the inlet 122 through the intake tube 148. In this arrangement, the pump 144 can cause a laminar flow of the gas through the chamber 254 between the inlet 122 and the outlet 124. As shown in FIG. 2C, the laminar flow of gas flows across the nasal access opening 140, forcing the droplets and/or the aerosols exhaled by the wearer 112 away from the nasal access opening 140 and towards the outlet 124. The outlet 124 then exhausts out of the chamber 254 the gas along with the droplets and/or the aerosols exhaled by the wearer 112 while wearing the mask 110. In FIG. 2C, the pump 144 includes the gas filter 150 that can remove the droplets and/or the aerosols from the gas prior to the gas exiting the pump 144.

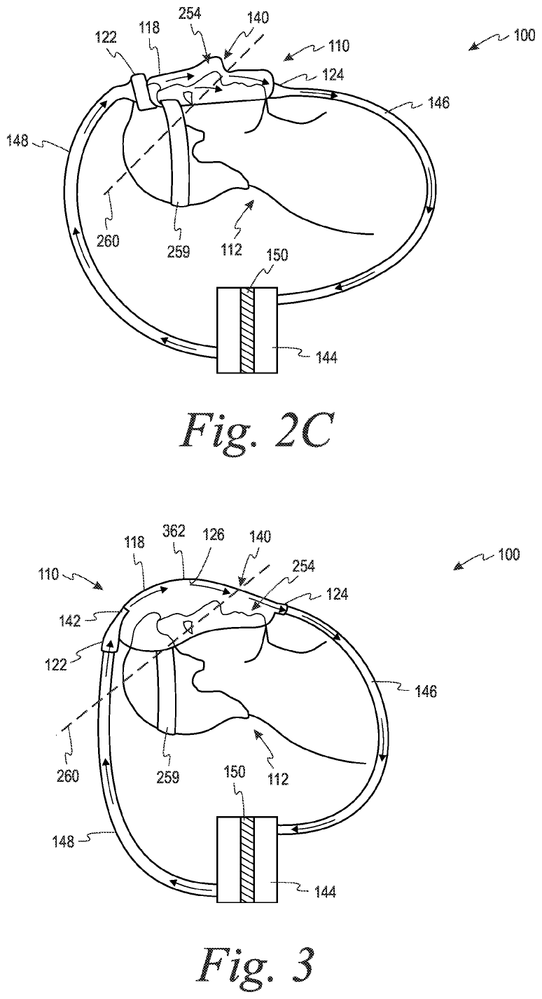

FIG. 3 show the surgical mask system 100 for performing a medical procedure via the nasal cavity according to another example implementation. In FIG. 3, the mask 110 and the pump 144 are substantially similar or identical to the mask 110 and the pump 144 shown in FIG. 2C, except the containment wall 118 of the mask 110 is differently shaped and the inlet 122 includes the nozzle 142 with an inwardly tapering shape in FIG. 3.

For example, in FIG. 3, the mask 110 includes the containment wall 118, the gasket 120 (shown in FIG. 1), the inlet 122, the outlet 124, and the nasal access opening 140 as described above. When the mask 110 is worn by the wearer 112 (e.g., when the mask 110 is coupled to the wearer by the strap 259), the nasal access opening 140 is aligned with the nose of the wearer 112 (e.g., aligned with the axis 260 of the nostrils and/or the nasal cavities of the wearer 112) and provides access for an instrument to the nasal cavities of the wearer 112. Additionally, in FIG. 3, the outlet 124 is coupled to the pump 144 by the exhaust tube 146 and/or the inlet 122 is coupled to the pump 144 by the intake tube 148. The pump 144 can also include the gas filter 150 to remove one or more particles (e.g., the droplets and/or aerosols) from the gas flowing into the chamber 254 and/or remove one or more particles from the gas exhausted out of the chamber 254.

As shown in FIG. 3, the inner surface 126 of the containment wall 118 has a rounded shape with an apex 362 that is located between the inlet 122 and the nasal access opening 140. The rounded shape of the inner surface 126 can omit any sharp corners and, thus, help to provide the laminar flow of the gas between the inlet 122 and the outlet 124. Additionally, the rounded apex 362 can help to align a direction of the laminar flow of the gas with the outlet 124 at the nasal access opening 140. This can help to provide a relatively direct and short flow path between the nose and the mouth of the wearer 112 and the outlet 124, which can help to efficiently and effectively direct the droplets and/or the aerosols to the outlet 124.

Additionally, in FIG. 3, the nozzle 142 has cross-sectional dimensions that gradually taper inwardly along a direction of flow of the gas through the nozzle 142. This can help to increase the flow rate of the gas while maintaining the laminar flow with a relatively smooth stream of the gas at the inlet 122. Additionally, to assist in producing the laminar flow of the gas, the nozzle 142 is oriented such that the gas exits the nozzle 142 substantially parallel to the inner surface 126 of the containment wall 118.

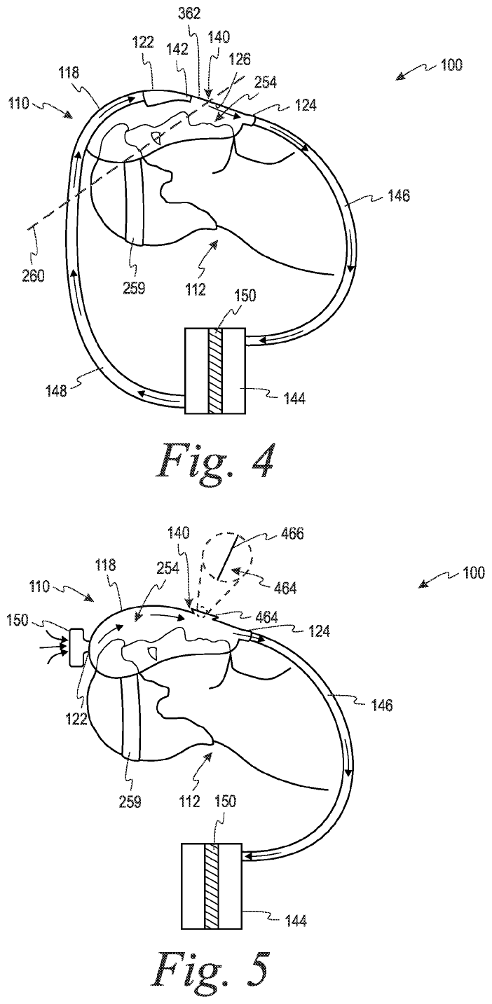

FIG. 4 show the surgical mask system 100 for performing a medical procedure via the nasal cavity according to another example implementation. In FIG. 4, the mask 110 and the pump 144 are substantially similar or identical to the mask 110 and the pump 144 shown in FIG. 3, except the nozzle 142 is located at the apex 362 in FIG. 4. As such, the nozzle 142 can be oriented to provide the gas along a substantially straight flow path between the inlet 122 and the outlet 124 (e.g., without any curves, turns, or corners). This can help to provide the laminar flow of the gas, and efficiently and effectively direct the droplets and/or the aerosols exhaled by the wearer 112 to the outlet 124.

FIG. 5 show the surgical mask system 100 for performing a medical procedure via the nasal cavity according to another example implementation. In FIG. 5, the mask 110 and the pump 144 are substantially similar or identical to the mask 110 and the pump 144 shown in FIG. 3, except the pump 144 is not coupled to the inlet 122 and the mask 110 includes a membrane 464 at the nasal access opening 140.

For example, in FIG. 5, the mask 110 includes the containment wall 118, the gasket 120 (shown in FIG. 1), the inlet 122, the outlet 124, and the nasal access opening 140 as described above. When the mask 110 is worn by the wearer 112 (e.g., when the mask 110 is coupled to the wearer by the strap 259), the nasal access opening 140 is aligned with the nose of the wearer 112 and provides access for an instrument to the nasal cavities of the wearer 112.

As described above, the inlet 122 that can receive the gas into the chamber 254 and the outlet 124 that can exhaust the gas out of the chamber 254. In FIG. 5, the outlet 124 is coupled to the pump 144 by the exhaust tube 146, and the pump 144 can include the gas filter 150 to filter the gas exhausted out of the chamber 254.

As noted above, the inlet 122 is not coupled to the pump 144 and, thus, the pump 144 does not provide the positive flow of gas to the inlet 122 in FIG. 5. Instead, in the example shown in FIG. 5, the laminar flow of gas is assisted by only the suction provided at the outlet 124 by the pump 144. As shown in FIG. 5, the gas filter 150 can also be disposed at the inlet 122 to remove one or more particles from the gas as the gas is received into the chamber 254. Accordingly, as shown in FIG. 5, the gas filter 150 can include a plurality of gas filters in some implementations (e.g., a first gas filter in the pump 144 and a second gas filter at the inlet 122).

Additionally, as noted above, the mask 110 includes the membrane 464 covering the nasal access opening 140 in FIG. 5. The membrane 464 can include a membrane opening 466 that provides access to the nasal access opening 140. In general, the membrane 464 and/or the membrane opening 466 are configured such that a size and/or a position of the membrane opening 466 is adjustable. In this arrangement, the membrane 464 can help to inhibit egress of the droplets and/or the aerosols exhaled by the wearer 112 while providing access to the nose 116 and the nasal cavities via the membrane opening 466.

In some examples, the membrane 464 can be formed from an elastic material that allows the membrane opening 466 to (i) deform from a closed state to an enlarged state responsive to an instrument being inserted in the nasal access opening 140 and applying a force to the membrane 464, and (ii) return from the enlarged state to the closed state responsive to the instrument being withdrawn from the nasal access opening 140. Additionally or alternatively, the elastic material of the membrane 464 can provide for (i) temporarily moving the membrane opening 466 from an initial position to an adjusted position (relative to the containment wall 118) responsive to an instrument being inserted in the nasal access opening 140 and applying a force to the membrane 464, and (ii) returning the membrane opening 466 from the adjusted position to the initial position responsive to the instrument being withdrawn from the nasal access opening 140. As examples, the membrane 464 can be formed from one or more materials selected from among a group of materials including silicone, nylon, cotton, polyester, and a plastic film (e.g., polyethylene film or polyvinyl chloride film).

In some examples, the mask 110 can be configured to provide the laminar flow of the gas between the inlet 122 and the outlet 124 (and across the nasal access opening 140) as described above. In other examples, due to the relatively smaller size of the nasal access opening 140 provided by the membrane 464 and the membrane opening 466, the gas can flow between the inlet 122 and the outlet 124 without a laminar flow (e.g., with a turbulent flow) while still inhibiting or preventing egress of the droplets and/or the aerosols from the chamber 254.

In FIG. 5, the membrane opening 466 defines a slit that is oriented in a horizontal plane such that the slit extends across both nostrils of the wearer 112 when the mask 110 is worn by the wearer 112. The horizontal plane can be, for instance, a plane extending between the first lateral side of the containment wall 118 and the second lateral side of the containment wall 118, and perpendicular to a median plane that extends between the superior end 256 and the inferior end 258 (shown in FIGS. 2A-2B) of the containment wall 118. This horizontal orientation of the slit can help to reduce (or minimize) a resistance of the membrane 464 on the instrument for a range of motion of the instrument in the horizontal plane (e.g., while moving the instrument from one nostril to another nostril of the wearer 112). Additionally, for example, orienting the slit in the horizontal plane can help to provide for simultaneously inserting a plurality of instruments in a side-by-side arrangement through the nasal access opening 140 (e.g., to simultaneously insert an instrument and an endoscope in a side-by-side arrangement to perform a procedure using the instrument under endoscopic visualization).

Although the slit defined by the membrane opening 466 is oriented in the horizontal plane as shown in FIG. 5, the slit can be oriented differently in other examples. In another example, the slit can be oriented in the median plane (e.g., parallel to the direction extending between the superior end 256 and the inferior end 258 in FIGS. 2A-2B). This vertical orientation of the slit can help to reduce (or minimize) a resistance of the membrane 464 on the instrument for a range of motion of the instrument in the median plane. This may also help to provide for simultaneously inserting the plurality of instruments in a vertical arrangement (e.g., an arrangement in which an endoscope is positioned above or below an instrument to perform a procedure using the instrument under endoscopic visualization). In additional or alternative examples, the slit can be oriented transverse to the horizontal plane and the median plane. This can provide for alternative ranges of motion of the instrument relative to one or more of the nostrils of the wearer 112.

Additionally, although the membrane opening 466 is a slit in FIG. 5, the membrane opening 466 can have a different shape in other examples. As examples, the membrane opening 466 can have a circular shape, an oval shape, a V-shape, a star shape, an X shape, and/or a bow-tie shape. Also, although the membrane 464 includes a single membrane opening 466 in FIG. 5, the membrane 464 can include a plurality of membrane openings 466 in other examples. For instance, the membrane 464 can include a first membrane opening 466 for accessing a first nostril and a second membrane opening 466 for accessing a second nostril, where a portion of the membrane 464 extends between the first membrane opening 466 and the second membrane opening 466. This can allow for reducing (or minimizing) a total size of the membrane openings 466 relative to a single membrane opening 466 that provides equivalent access to both nostrils. In an example, the quantity, the size, the shape, and/or the orientation of the membrane opening 466 can be based on a procedure that is to be performed using the mask 110 and/or a location of a target tissue in the nasal cavity of the wearer 112 that will be accessed during the procedure.

As described above, the membrane 464 can cover the nasal access opening 140. In one example, the membrane 464 can be coupled to the outer surface 128 (FIGS. 1 and 2A) of the containment wall 118. In another example, the membrane 464 can be additionally or alternatively coupled to the inner surface 126 (FIGS. 1 and 2B) of the containment wall 118. In another example, the membrane 464 can be additionally or alternatively coupled to the edge of the containment wall 118 that defines the nasal access opening 140 between the inner surface 126 of the containment wall 118 and the outer surface 128 of the containment wall 118.

In some implementations, the membrane 464 can be non-removably coupled to the containment wall 118. This may help to simplify and/or reduce a cost of manufacture. Additionally, non-removably coupling the membrane 464 to the containment wall 118 at a time of manufacture can help to simplify preparing the surgical mask system 100 for use relative to implementations in which the membrane 464 is coupled to the containment wall 118 by a practioner prior to a procedure. As examples, the membrane 464 can be coupled to the containment wall 118 by at least one coupling selected from among a group consisting of: an adhesive and a

In other implementations, the membrane 464 can be removably coupled to the containment wall 118 such that the membrane 464 can be coupled, decoupled, and recoupled to the containment wall 118. This can allow for a medical practioner to select a membrane 464 from among a plurality of membranes 464 and couple the selected membrane 464 to the containment wall 118 at the nasal access opening 140. For instance, the membrane openings 466 of the plurality of membranes 464 can have different configurations (e.g., quantity, size, shape, and/or orientation) such that the medical practioner can select the membrane 464 that is well suited for a particular patient's anatomy and/or a particular type of procedure to be performed.

As examples, the membrane 464 can be removably coupled to the containment wall 118 at the nasal access opening 140 by a friction fit engagement between the membrane 464 and the edge of the containment wall 118. As such, the membrane 464 can have a shape and a size that approximately corresponds to a shape and a size of the nasal access opening 140 defined by the containment wall 118. Additionally, when the membrane 464 is formed from an elastic material, the elasticity of the membrane 464 can help to frictionally engage the membrane 464 with the edge of the containment wall 118.

In some examples, the membrane 464 can be formed from a single layer of material. For instance, the membrane 464 can be a single, monolithic piece of material that is non-removably or removably coupled to the containment wall 118 at the nasal access opening 140. This can help to simplify and/or reduce a cost of manufacturing of the mask 110.

In other examples, the membrane 464 can include a plurality of layers of material. This can provide one or more of the technical benefits described in further detail below.

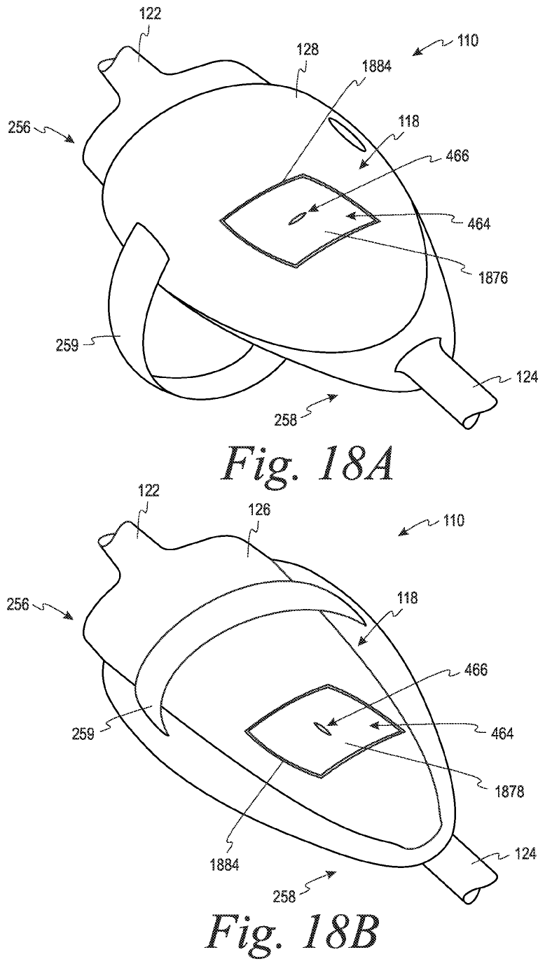

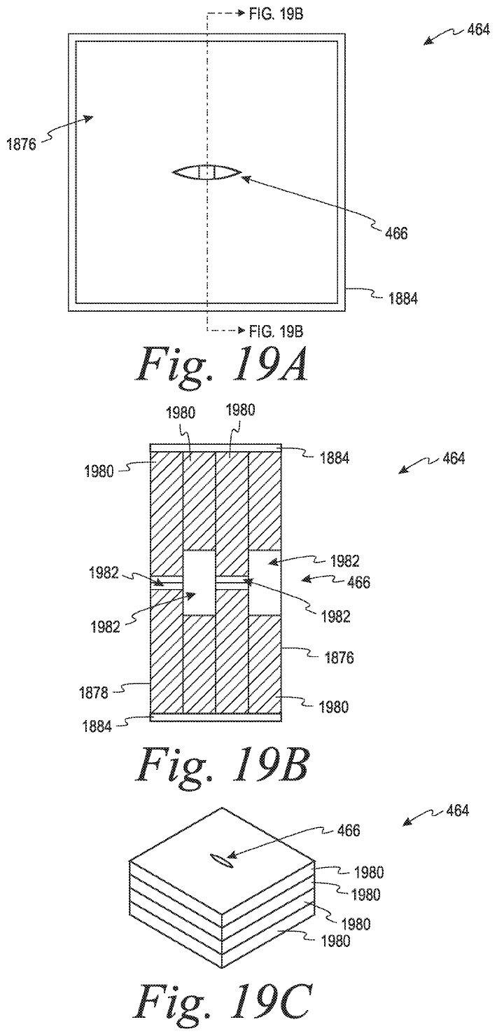

FIGS. 18A-19C depict an implementation of the surgical mask system 100 shown in FIGS. 1 and 5 in which the membrane 464 includes a plurality of layers 1980 of material, according to an example. More particularly, FIG. 18A depicts a perspective view of an outer surface 1876 of the mask 110 including the membrane 464, and FIG. 18B shows a perspective view of an inner surface 1878 of the mask 110 shown in FIG. 18A. Additionally, FIG. 19A depicts a top view of the outer surface 1876 of the membrane 464 shown in FIG. 18A, FIG. 19B depicts a cross-sectional view of the membrane 464 taken through a line shown in FIG. 19A, and FIG. 19C shows a partial perspective view of the layers 1980 of the membrane 464.

As shown in FIGS. 18A-18B, the membrane 464 extends across the nasal access opening 140 defined by the containment wall 118 of the mask 110. In this way, the membrane 44 can provide a barrier for inhibiting egress of the droplets and/or the aerosols exhaled by the wearer 112. As described above, the membrane 464 can be removably coupled or non-removably coupled to the containment wall 118 at the nasal access opening 140.

In FIGS. 18A-19A, the membrane 464 has a square shape that matches a corresponding square shape of the containment wall 118 at the nasal access opening 140. However, in other examples, the membrane 464 and the nasal access opening 140 can have a different shape such as, for instance, a circle shape, an oval shape, a rectangle shape, a triangle shape, a star shape, other polygonal shapes, or other non-polygonal shapes. In some implementations, the shape of the membrane 464 and the nasal access opening 140 can be configured to assist in coupling the membrane 464 to the containment wall 118 with the membrane opening 466 in a predefined position and/or a predefined orientation relative to the containment wall 118. For instance, the shape of the membrane 464 and the nasal access opening 140 can be a non-circular shape and/or an asymmetric shape such that the membrane 464 is couplable to the containment wall 118 at the nasal access opening 140 only when with the membrane opening 466 is in the predefined position and/or the predefined orientation.

As shown in FIGS. 18A-19C, the membrane 464 includes the outer surface 1876 (shown in FIG. 18A), the inner surface 1878 (shown in FIG. 18B), and a plurality of layers 1980 (shown in FIGS. 19B-19C) in a stacked arrangement between the outer surface 1876 and the inner surface 1878 of the membrane 464. Similar to the inner surface 126 and the outer surface 128 of the containment wall 118, the inner surface 1878 of the membrane 464 is configured to extend over the face 114 of the wearer 112 and the outer surface 1876 of the membrane 464 is opposite the inner surface 1878 of the membrane 464. In this arrangement, the membrane 464 can cooperate with the containment wall 118 and the gasket 120 to define the chamber (shown in FIG. 5) between (i) the inner surface 126 of the containment wall 118 and the inner surface 1878 of the membrane 464 and (ii) the face 114 of the wearer 112 when the mask 110 is worn by the wearer 112.

As shown in FIG. 19B, each layer 1980 includes a respective aperture 1982, and the respective apertures 1982 of the layers 1980 overlap with each other to define the membrane opening 466 extending through the membrane 464 between the outer surface 1876 of the membrane 464 and the inner surface 1878 of the membrane 464.

In some examples, the apertures 1982 can have the same size, the same shape, the same orientation, and the same position in the respective layers 1980 relative to each other. As a result, the membrane opening 466 can have a consistent cross-sectional shape through an entire thickness of the membrane 464 between the outer surface 1876 and the inner surface 1878. This can provide for forming the membrane opening 466 in any one of the configurations described above with respect to FIG. 5 (e.g., the membrane opening 466 having a slit shape, a circular shape, an oval shape, a V-shape, a star shape, an X shape, and/or a bow-tie shape).

In other examples, the aperture 1982 of at least one layer 1980 can differ from the aperture 1982 of at least one other layer 1980 in at least one characteristic selected from among: a size, a shape, an orientation, and a position. This can, for instance, help the membrane 464 to more fine-tuned control over an interaction between the instrument and the membrane 464 when the instrument is inserted through and/or withdrawn from the membrane opening 466. Additionally, for example, providing the layers 1980 with the apertures 1982 having different configurations can help to reinforce the membrane 464 against tearing (e.g., tearing at endpoints of a slit).

As an example, in FIG. 19A-19C, each aperture 1982 defines a slit, and the slits of adjacent layers 1980 in the stacked arrangement are elongated in respective directions that are transverse to each other. This can, for instance, help the membrane 464 to engage a greater amount of a circumference of instrument extending through the membrane opening 466 than implementations in which the membrane 464 is formed from a single layer of material and have a similar range of motion relative to the containment wall 118. By contacting a greater surface area around the instrument, the membrane 464 can assist in physically removing virus and infectious bacteria from the instrument as the instrument is withdrawn from the membrane opening 466.

For instance, as shown in FIGS. 19A-19B, the respective directions in which the slits of the adjacent layers 1980 are elongated are arranged at an angle of approximately 90 degree relative to each other. This can help to an enhance an extent to which the membrane opening 466 can expand open relative to other implementations having different angles at which the slits are arranged relative to each other. Additionally, enhancing the extent to which the membrane opening 466 can expand can help to provide access to relatively large instruments.

In another example implementation, the respective directions in which the slits of the adjacent layers 1980 are elongated are arranged at an angle between approximately 45 degrees and approximately 60 degrees relative to each other. This can help to more effectively seal the membrane 464 around an instrument relative to the implementation in which the adjacent slits are perpendicular to each other. Additionally or alternatively, the angle of slits relative to each other can be selected based on, for instance, a range of motion for performing a procedure, a shape of the instrument, and/or a sealing performance of the membrane 464.

In one example in which the apertures 1982 are slits, each slit can have a length between approximately 5 millimeters (mm) and approximately 40 mm, where the length is a dimension along which the slit is elongated. This length can help to accommodate many (if not all) instruments that may be inserted through and move within the membrane opening 466 to access and perform a procedure in the nasal cavity of the wearer 112 of the mask 110. In one example, a length of each slit can be selected based on at least one criteria selected from a group consisting of: a quantity of layers 1980, one or more material(s) of membrane 464, a surface area of membrane 464, a thickness of membrane 464, a quantity of membrane openings 466, a shape of membrane 464, and one or more instrument(s) that are intended to be passed through the membrane opening 466.

In some examples, the membrane 464 can also include a membrane gasket 1884 that is configured to couple the membrane 464 to the containment wall 118 at the nasal access opening 140. For instance, as shown in FIGS. 18A-19B, the membrane gasket 1884 can extend around an entire periphery of the membrane 464, and the layers 1980 can be coupled to the membrane gasket 1884. As a result, the membrane gasket 1884 can help to retain the layers 1980 of material in the stacked arrangement (e.g., the membrane gasket 1884 can help to mitigate the layers 1980 separating at the periphery). Additionally, the membrane gasket 1884 can provide a structural support with greater rigidity than the layers 1980 of material to assist in coupling the membrane 464 to the containment wall 118 at the nasal access opening 140 as described above (e.g., via a friction fit coupling and/or an adhesive coupling).

In some examples, the membrane 464 can include an anti-viral material, and the membrane 464 can be configured to transfer at least a portion of the anti-viral material to the instrument responsive to the instrument applying the force to the membrane 464 in the membrane opening 466. In such examples, when the instrument is withdrawn through the membrane opening 466, the instrument can apply the force to the membrane 464 causing the anti-viral material to transfer to the instrument and deactivate any virus that may have attached to the instrument while in the chamber.

In some examples, the anti-viral material can be disposed between adjacent layers 1980 in the stacked arrangement. In one implementation, the anti-viral material can be disposed between the adjacent layers 1980 while manufacturing the membrane 464 (e.g., while positioning the layers 1980 into the stacked arrangement). In another implementation, the anti-viral material can be provided in a separate container and applied to the layers of material just prior to use of the mask 110. As examples, the anti-viral material can include one or more materials selected from among a group consisting of: silver nanoparticles, zinc oxide, copper, a polymer having anti-viral properties, and a biopolymer having anti-viral properties (e.g., chitosan).

In examples in which the membrane 464 includes the plurality of layers 1980, the layers 1980 can be formed from one or more materials selected from a group consisting of: (i) a fibrous material and (ii) an elastomeric material. The fibrous material may be beneficial in implementations in which the membrane 464 includes the anti-viral material as the fibrous material may facilitate absorption and/or retention of the anti-viral material on or between the layers 1980 of material of the membrane 464. The elastomeric material may be beneficially allow the membrane opening 466 to (i) deform from a closed state to an enlarged state responsive to an instrument being inserted through the membrane opening 466 and applying a force to the membrane 464, and (ii) return from the enlarged state to the closed state responsive to the instrument being withdrawn from the membrane 464. Additionally or alternatively, the layers 1980 can be formed from one or more materials selected from a group consisting of: silicone, nylon, cotton, polyester, and a plastic film (e.g., polyethylene film or polyvinyl chloride film).

In some implementations, the layers 1980 can all be formed from the same material. This may help to, for example, simplify a manufacturing process. In other implementations, different layers 1980 of the membrane 1980 can be formed from different materials (e.g., at least one layer 1980 can be made from a different material than at least another one of the layers 1980). As one example, a membrane 464 can include one or more layers 1980 formed from an elastomeric material to enhance sealing the membrane 464 around an instrument, and one or more layers 1980 formed from a fibrous material to enhance delivering an anti-viral material to the instrument.

Additionally, in some examples, the layers 1980 can be made from a material that causes the membrane 464 to be at least translucent. This can help to allow a practitioner to see the nasal cavity of the wearer 112 while the mask 110 is positioned on the face 114 of the wearer 112. For instance, as noted above, the layers 1980 can be formed from a translucent silicone and/or vinyl material.

In FIGS. 19B-19C, the membrane 464 includes four layers 1980. However, in other examples, the membrane 464 can include a different quantity of layers 1980. For instance, the plurality of layers 1980 can include a quantity of layers between two layers and five layers. This can help to achieve relatively good contact and sealing between the membrane 464 and an instrument to remove viruses from the instrument as described above.

Although the membrane 464 and the membrane opening 466 are shown in the example implementations of FIGS. 5 and 18A-19C, the membrane 464 and the membrane opening 466 can be included in any of the example implementations shown and described with respect to FIGS. 1-4.

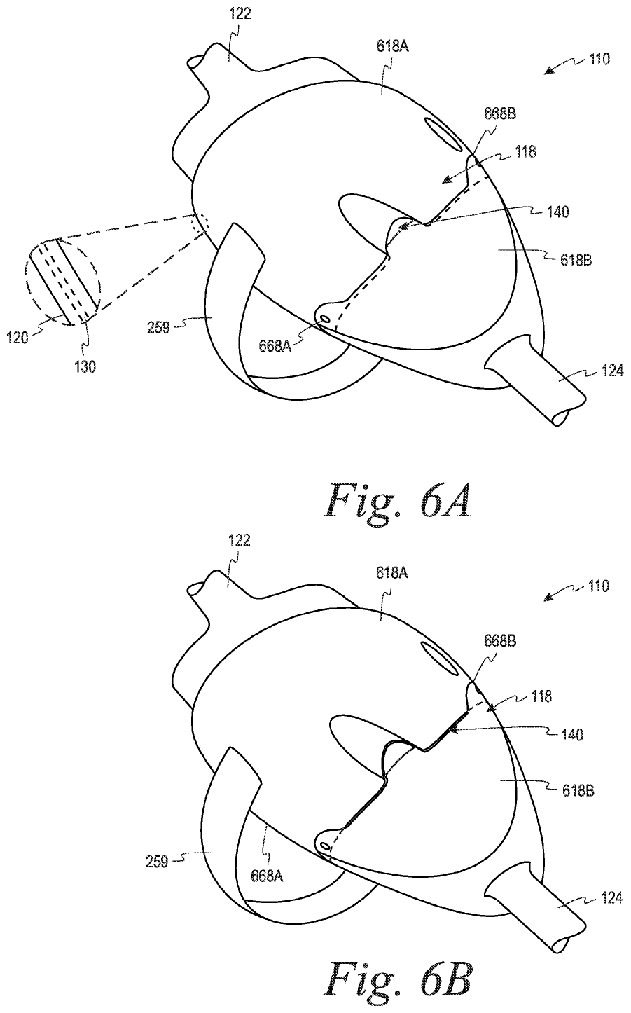

FIGS. 6A-6B show the mask 110 for performing a medical procedure via the nasal cavity according to another example implementation. In FIG. 6A-6B, the mask 110 is substantially similar or identical to the mask 110 shown in FIGS. 2A-2B, except the mask 110 is configured to change a size of the nasal access opening 140. This can help to tailor the size of the nasal access opening 140 to a particular anatomy of the wearer 112 as different wearers may have differently sized and/or differently shaped faces and/or noses.

For example, in FIGS. 6A-6B, the mask 110 includes the containment wall 118, the gasket 120 at the peripheral edge 130, the inlet 122, the outlet 124, and the nasal access opening 140 as described above. When the mask 110 is worn by the wearer 112 (e.g., when the mask 110 is coupled to the wearer by the strap 259), the nasal access opening 140 is aligned with the nose of the wearer 112 and provides access for an instrument to the nasal cavities of the wearer 112.

As shown in FIGS. 6A-6B, the containment wall includes a first section 618A and a second section 618B, and the first section 618A and the second section 618B are movable relative to each other to adjust a size of the nasal access opening 140. For example, the first section 618A and the second section 618B can be coupled to each other by a first hinge 668A and a second hinge 668B. In this arrangement, the first section 618A can hingedly move relative to the second section 618B between a plurality of positions relative to each other.

FIG. 6A depicts the mask 110 with the first section 618A and the second section 618B in a first position, and FIG. 6B depicts the mask 110 with the first section 618A and the second section 618B in a second position. As shown in FIGS. 6A-6B, the nasal access opening 140 has a first size when the first section 618A and the second section 618B are in the first position, the nasal access opening 140 has a second size when the first section 618A and the second section 618B are in the second position, and the second size is greater than the first size. Although FIGS. 6A-6B depict two example positions for the first section 618A and the second section 618B relative to each other, the first section 618A and the second section 618B can be in a plurality of other positions to controllably adjust the size of the nasal access opening 140.

In some examples, the first section 618A and the second section 618B can partially overlap with each other in one or more of the plurality of positions. For instance, at least a portion of the inner surface 126 of the first section 618A can overlap with at least a portion of the outer surface 128 of the second section 618B, or at least a portion the outer surface 128 of the first section 618A can overlap with at least a portion of the inner surface 126 of the first section 618A. This can help to limit the size of the nasal access opening to a region that is aligned with the nose 116 of the wearer 112 and, thus, help to inhibit the droplets and/or the aerosols from egressing from the chamber 254 through the nasal access opening 140.

In FIGS. 6A-6B, the first section 618A is a superior portion of the containment wall 118 and the second section 618B is an inferior portion of the containment wall 118. In some examples, the first section 618A can cover the forehead and at least a portion of the nose of the wearer, whereas the second section 618B can cover the mouth and chin of the wearer. However, the first section 618A and the second section 618B can cover alternative combinations of anatomical structures of the wearer 112 in other examples.

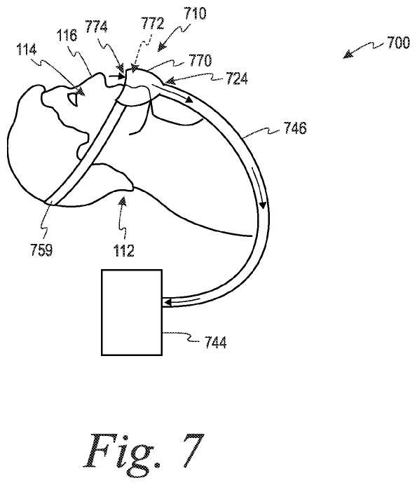

Referring now to FIG. 7, a surgical mask system 700 is shown according to an alternative example. The surgical mask system 700 differs from the surgical mask system 100 described above in that the surgical mask system 700 does not include the containment wall 118 that provides a physical barrier that helps to contain the droplets and/or aerosols exhaled by the wearer 112 within the chamber 254. Instead, the surgical mask system 700 is entirely open above at least the mouth of the wearer 112, and provides a higher pressure of suction (compared to the surgical mask system 100) to divert the droplets and/or the aerosols from the environment in which the medical practitioner is located.

As shown in FIG. 7, the surgical mask system 700 includes a mask 710, an exhaust tube 746, and a pump 714. The mask 710 includes a mask wall 770 configured to be positioned over a lower portion of a face 114 of a wearer 112 below a nose 116 of the wearer 112. The mask wall 770 defines a cavity 772 between the mask wall 770 and the face 114 of the wearer 112 when the mask 710 is worn by the wearer 112. Accordingly, the mask wall 770 can have a shape and a size that provides for a gap between the mask wall 770 and the face 114 of the wearer 112 when the mask 710 is positioned on the lower portion of the face 114 of the wearer 112.

As shown in FIG. 7, the mask 710 also includes an intake 774 at a superior end of the mask wall 770, and an outlet 724 at an inferior end of the mask wall 770. The intake 774 is configured to receive air along with droplets and/or aerosols exhaled by the wearer 112 into the cavity 772, and the outlet 724 is configured to exhaust the air along with the droplets and/or the aerosols out of the cavity 772. The exhaust tube 746 is coupled to the outlet 724 of the mask 710, and the pump 744 is coupled to the exhaust tube 746.

In this arrangement, the pump 744 is configured to provide suction at the outlet 724 to draw the air along with the droplets and/or the aerosols into the cavity 772 of the mask 710 and through the exhaust tube 746 to the pump 744 (and/or a gas filter and/or a waste container such as the gas filter 150 and/or the waste container 152 described above). The pump 744 can be substantially similar or identical to the pump 144 described above, except the pump 744 is operable to generate the suction at a relatively higher pressure than may be generated for the laminar flow of the gas described above due to the mask 710 not providing the substantially enclosed chamber 254 that is provided by the mask 110 described above.

Additionally, as shown in FIG. 7, the mask 710 can include a strap 759 that is configured to couple the mask 710 to the face 114 of the wearer 112. For example, similar to the strap 259 described above with respect to FIGS. 2A-6B, the strap 759 shown in FIG. 7 can extend around the head of the wearer 112 to maintain the mask 710 in a desired position on the face 114 of the wearer 112. Although the mask 710 includes the strap 759 in FIG. 7, the mask 710 can additionally or alternatively include another type of securement apparatus such as any of the types of apparatuses described above with respect to the securement apparatus 138.

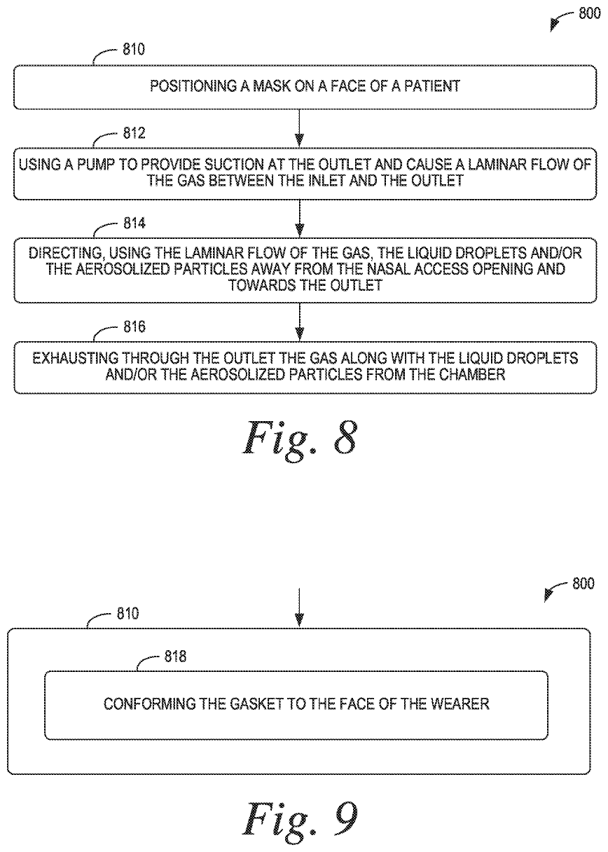

Referring now to FIG. 8, a flowchart for a process 800 of operating a surgical mask system is depicted according to an example. At block 810, the process 800 includes positioning a mask on a face of a patient. The mask can include a containment wall, a gasket, a nasal access in the containment wall, an inlet at a superior end of the containment wall, and an outlet at an inferior end of the containment wall. The containment wall can include an inner surface that extends over a face of a wearer, an outer surface opposite the inner surface, and a peripheral edge at an interface between the inner surface and the outer surface. The gasket is at the peripheral edge of the containment wall. The containment wall and the gasket define a chamber between the inner surface of the containment wall and the face of the wearer. The nasal access opening is aligned with a nose of the wearer. The inlet is configured to receive a gas into the chamber. The outlet is configured to exhaust out of the chamber the gas along with droplets and/or aerosols exhaled by the wearer while wearing the mask.

At block 812, the process 800 can include using a pump to provide suction at the outlet and cause a laminar flow of the gas between the inlet and the outlet. At block 814, the process 800 includes directing, using the laminar flow of the gas, the droplets and/or the aerosols away from the nasal access opening and towards the outlet. At block 816, the process 800 includes exhausting through the outlet the gas along with the droplets and/or the aerosols from the chamber.

FIGS. 9-17 depict additional aspects of the process 800 according to further examples. As shown in FIG. 9, positioning the mask on the face of the patient at block 810 can include conforming the gasket to the face of the wearer at block 818.

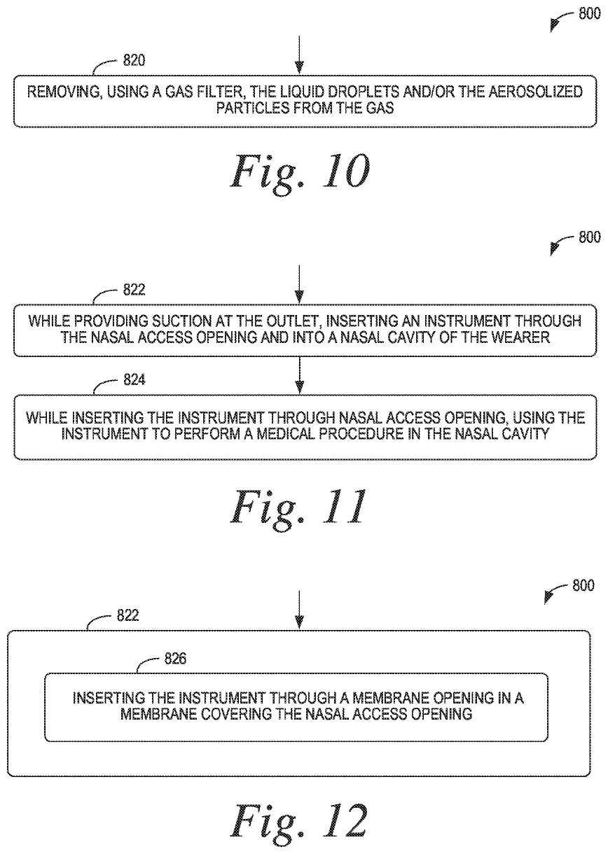

As shown in FIG. 10, the process 800 can further include removing, using a gas filter, the droplets and/or the aerosols from the gas at block 820.

As shown in FIG. 11, the process 800 can also include: (i) while providing suction at the outlet, inserting an instrument through the nasal access opening and into a nasal cavity of the wearer at block 822, and (ii) while inserting the instrument through nasal access opening, using the instrument to perform a medical procedure in the nasal cavity at block 824.

As shown in FIG. 12, inserting the instrument through the nasal access opening at block 822 can include inserting the instrument through a membrane opening in a membrane covering the nasal access opening at block 826

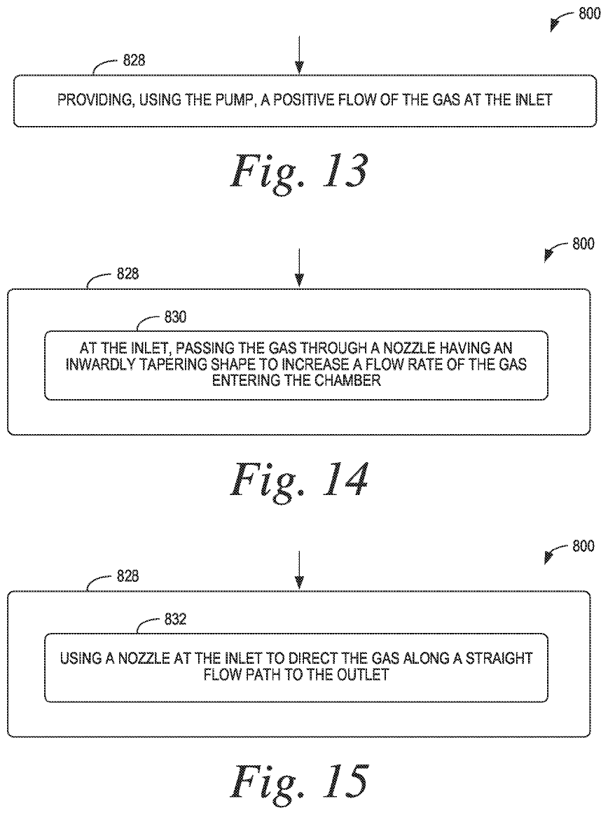

As shown in FIG. 13, the process 800 can further include providing, using the pump, a positive flow of the gas at the inlet at block 828.

As shown in FIG. 14, providing the positive flow of the gas at the inlet at block 828 can include, at the inlet, passing the gas through a nozzle having an inwardly tapering shape to increase a flow rate of the gas entering the chamber at block 830.

As shown in FIG. 15, providing the positive flow of the gas at the inlet at block 828 can include using a nozzle at the inlet to direct the gas along a straight flow path to the outlet at block 832.

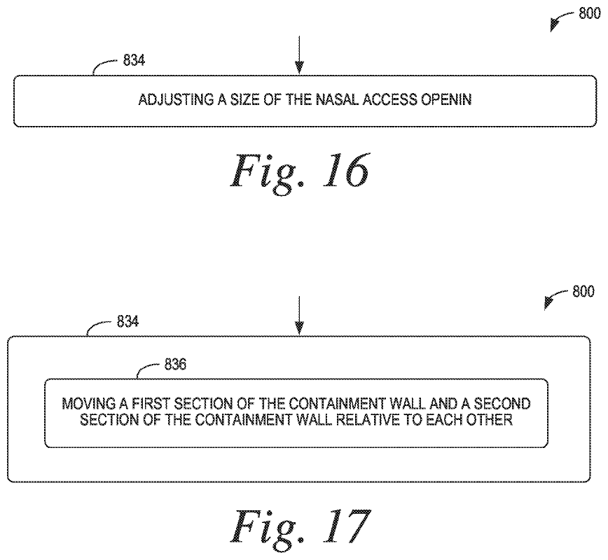

As shown in FIG. 16, the process 800 can also include adjusting a size of the nasal access opening at block 834.

As shown in FIG. 17, adjusting the size of the nasal access opening at block 830 can include moving a first section of the containment wall and a second section of the containment wall relative to each other at block 836.

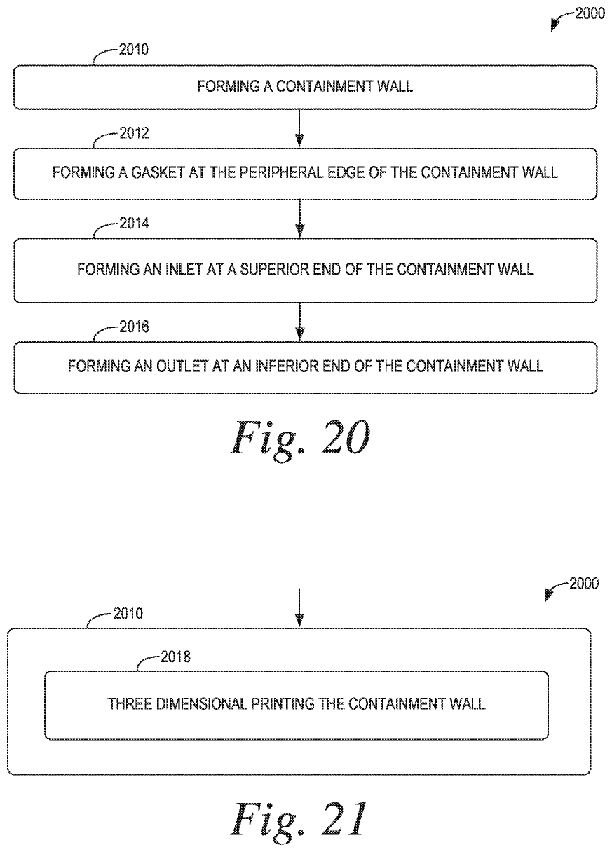

Referring now to FIG. 20, a flowchart of a process 2000 for forming a mask for performing a medical procedure via a nasal cavity is shown according to an example. As shown in FIG. 20, at block 2010, the process 2000 can include forming a containment wall. The containment wall can include: (i) an inner surface configured to extend over a face of a wearer, (ii) an outer surface opposite the inner surface, (iii) a peripheral edge at an interface between the inner surface and the outer surface, and (iv) a nasal access opening that is configured to be aligned with a nose of the wearer when the mask is worn by the wearer.

At block 2012, the process 2000 can include forming a gasket at the peripheral edge of the containment wall. The gasket can be configured to conform to the face of a wearer. The containment wall and the gasket define a chamber between the inner surface of the containment wall and the face of the wearer when the mask is worn by the wearer.

At block 2014, the process 2000 can include forming an inlet at a superior end of the containment wall. The inlet can be configured to receive a gas into the chamber. At block 2016, the process 2000 can include forming an outlet at an inferior end of the containment wall. The outlet can be configured to exhaust out of the chamber (i) the gas and (ii) at least one of droplets or aerosols exhaled by the wearer while wearing the mask.

FIGS. 21-30 depict additional aspects of the process 2000 according to further examples. As shown in FIG. 21, forming the containment wall at block 2010 can include three dimensional printing the containment wall at block 2018.

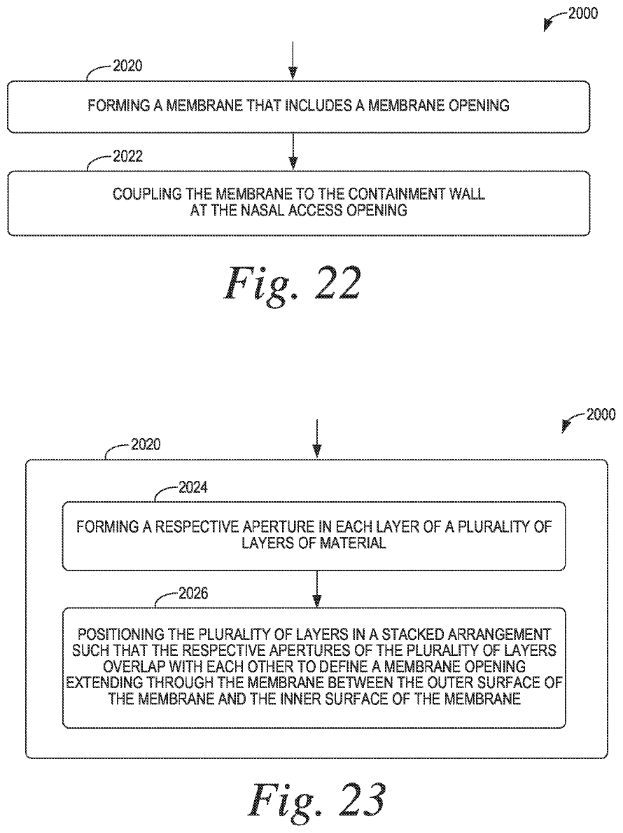

As shown in FIG. 22, the process 2000 can further include forming a membrane that includes a membrane opening at block 2020, and coupling the membrane to the containment wall at the nasal access opening at block 2022. The membrane can cover the nasal access opening when the membrane is coupled to the containment wall at the nasal access opening.

As shown in FIG. 23, forming the membrane at block 2020 can include forming a respective aperture in each layer of a plurality of layers of material at block 2024, and positioning the plurality of layers in a stacked arrangement such that the respective apertures of the plurality of layers overlap with each other to define a membrane opening extending through the membrane between the outer surface of the membrane and the inner surface of the membrane at block 2026.

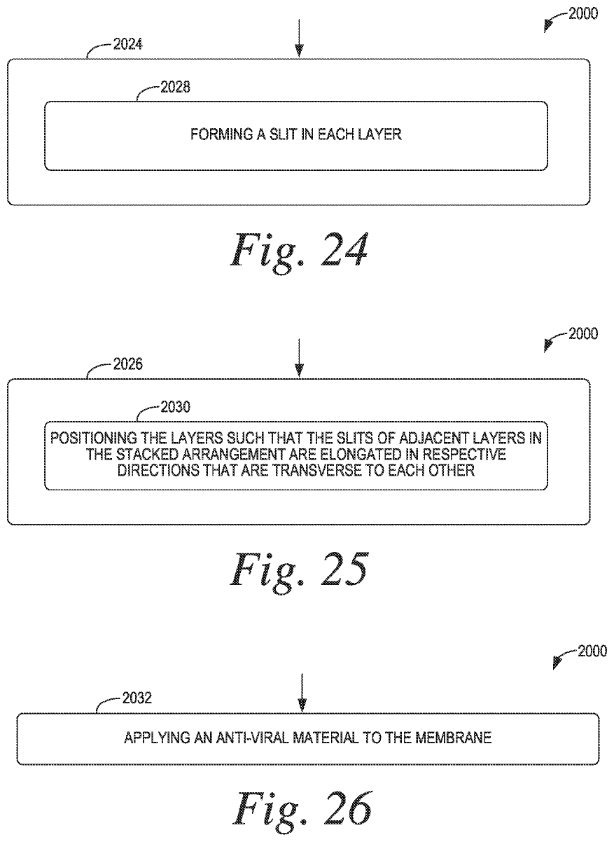

As shown in FIG. 24, forming the respective aperture in each layer at block 2024 can include forming a slit in each layer at block 2028.

As shown in FIG. 25, positioning the plurality of layers in the stacked arrangement at block 2026 can include positioning the layers such that the slits of adjacent layers in the stacked arrangement are elongated in respective directions that are transverse to each other at block 2030.

As shown in FIG. 26, the process 2000 can also include applying an anti-viral material to the membrane at block 2032. The membrane can be configured to transfer at least a portion of the anti-viral material to an instrument responsive to the instrument applying a force to the membrane in the membrane opening. Within examples, applying the anti-viral material to the membrane at block 2032 can be performed before, during, or after positioning the plurality of layers in the stacked arrangement at block 2026.

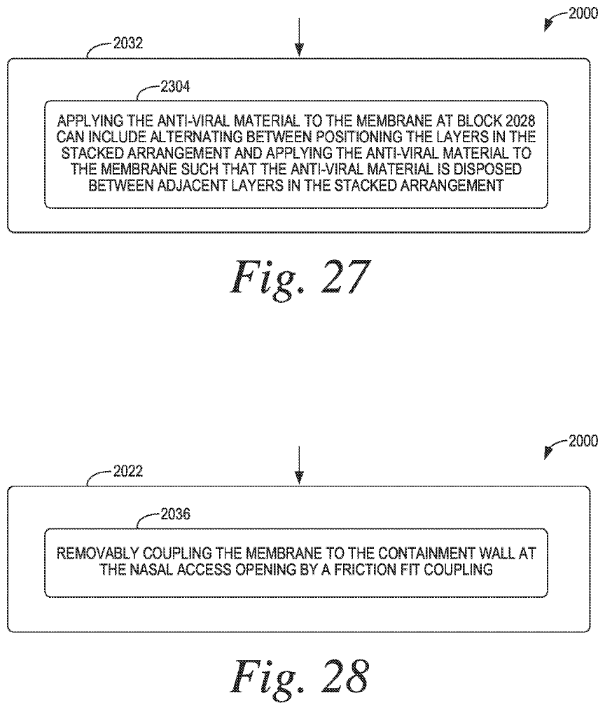

As shown in FIG. 27, applying the anti-viral material to the membrane at block 2032 can include alternating between positioning the layers in the stacked arrangement and applying the anti-viral material to the membrane such that the anti-viral material is disposed between adjacent layers in the stacked arrangement at block 2034.

As shown in FIG. 28, coupling the membrane to the containment wall at block 2022 can include removably coupling the membrane to the containment wall at the nasal access opening by a friction fit coupling at block 2036.

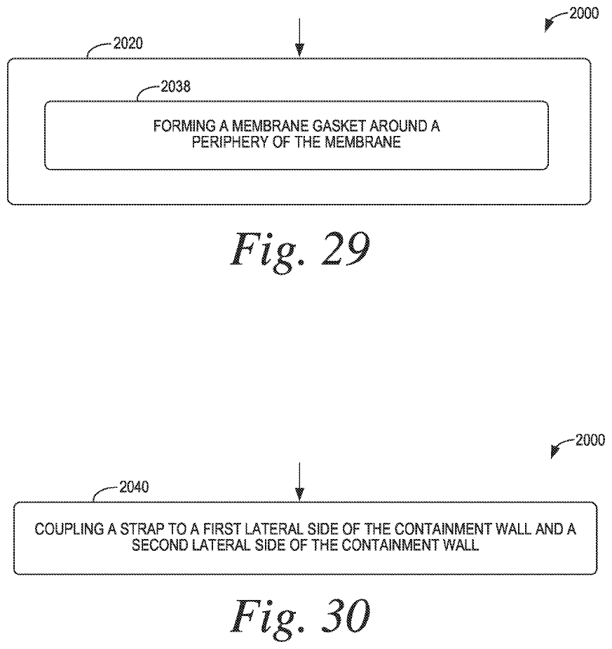

As shown in FIG. 29, forming the membrane at block 2020 can further include forming a membrane gasket around a periphery of the membrane at block 2038.

As shown in FIG. 30, the process 2000 can also include coupling a strap to a first lateral side of the containment wall and a second lateral side of the containment wall at block 2040. The strap can be configured to couple the mask to a head of the wearer.

The description of the different advantageous arrangements has been presented for purposes of illustration and description, and is not intended to be exhaustive or limited to the embodiments in the form disclosed. Many modifications and variations will be apparent to those of ordinary skill in the art. Further, different advantageous embodiments may describe different advantages as compared to other advantageous embodiments. The embodiment or embodiments selected are chosen and described in order to explain the principles of the embodiments, the practical application, and to enable others of ordinary skill in the art to understand the disclosure for various embodiments with various modifications as are suited to the particular use contemplated.

1. A mask for performing a medical procedure via a nasal cavity, comprising:
a containment wall comprising:
an inner surface configured to extend over a face of a wearer,
an outer surface opposite the inner surface, and
a peripheral edge at an interface between the inner surface and the outer surface;
a gasket at the peripheral edge of the containment wall, wherein the gasket is configured to conform to the face of a wearer, wherein the containment wall and the gasket define a chamber between the inner surface of the containment wall and the face of the wearer when the mask is worn by the wearer,
a nasal access opening in the containment wall, wherein the nasal access opening is aligned with a nose of the wearer when the mask is worn by the wearer;
an inlet at a superior end of the containment wall, wherein the inlet is configured to receive a gas into the chamber; and
an outlet at an inferior end of the containment wall, wherein the outlet is configured to exhaust out of the chamber (i) the gas and (ii) at least one of droplets or aerosols exhaled by the wearer while wearing the mask.

2. The mask of claim 1, wherein the containment wall is configured to provide a laminar flow of the gas between the inlet and the outlet, and
wherein the laminar flow of the gas directs the at least one of the droplets or the aerosols toward the outlet.

3. The mask of claim 2, wherein the inner surface of the containment wall has a rounded shape with an apex that is located between the inlet and the nasal access opening.

4. The mask of claim 2, wherein the laminar flow of the gas flows across the nasal access opening to inhibit the at least one of the droplets or the aerosols from flowing out of the chamber at the nasal access opening.

5. The mask of claim 2, wherein the laminar flow of the gas is over at least a central longitudinal portion of the containment wall such that the gas flows over the nose and a mouth of the wearer when the wearer wears the mask.

6. The mask of claim 1, further comprising a membrane covering the nasal access opening, and
wherein the membrane comprises a membrane opening that provides access to the nasal access opening.

7. The mask of claim 6, wherein the membrane has an outer surface and an inner surface,
wherein the membrane comprises a plurality of layers in a stacked arrangement between the outer surface and the inner surface of the membrane,
wherein each layer comprises a respective aperture,
wherein the respective apertures of the plurality of layers overlap with each other to define a membrane opening extending through the membrane between the outer surface of the membrane and the inner surface of the membrane.

8. The mask of claim 7, wherein each aperture defines a slit,
wherein the slits of adjacent layers in the stacked arrangement are elongated in respective directions that are transverse to each other.

9. The mask of claim 8, wherein the respective directions in which the slits of the adjacent layers are elongated are arranged at an angle of approximately 90 degree relative to each other.

10. The mask of claim 8, wherein the respective directions in which the slits of the adjacent layers are elongated are arranged at an angle between approximately 45 degrees and approximately 60 degrees relative to each other.

11. (canceled)

12. The mask of claim 7, wherein the plurality of layers comprise an elastic material such that the membrane opening is configured to (i) deform from a closed state to an enlarged state responsive to an instrument being inserted through the membrane opening and applying a force to the membrane, and (ii) return from the enlarged state to the closed state responsive to the instrument being withdrawn from the membrane opening.

13. The mask of claim 7, wherein the membrane comprises an anti-viral material, and
wherein the membrane is configured to transfer at least a portion of the anti-viral material to the instrument responsive to the instrument applying the force to the membrane in the membrane opening.

14. The mask of claim 13, wherein the anti-viral material is disposed between adjacent layers in the stacked arrangement.

15. (canceled)

16. The mask of claim 7, wherein the membrane is removably coupled to the containment wall at the nasal access opening by a friction fit coupling.

17. (canceled)

18. The mask of claim 7, wherein the membrane is made from a material that causes the membrane to be at least translucent.

19. The mask of claim 7, wherein the plurality of layers comprises a quantity of layers between two layers and five layers.

20. The mask of claim 1, wherein the containment wall comprises a first section and a second section, and wherein the first section and the second section are movable relative to each other to adjust a size of the nasal access opening.

21. The mask of claim 1, wherein the containment wall is made from a material that causes the containment wall to be at least partially translucent.

22. The mask of claim 1, further comprising a gas filter at the inlet, wherein the gas filter is configured to remove one or more particles from the gas as the gas is received into the chamber.

23. The mask of claim 1, further comprising a strap extending from a first lateral side of the containment wall to a second lateral side of the containment wall, wherein the strap is configured to couple the mask to a head of the wearer.

24-76. (canceled)
